(19) United States
(12) Patent Application Publication
(10) Pub. No.: US 2022/0328141 A1
(43) Pub. Date: Oct. 13, 2022

(54) SYSTEMS AND METHODS FOR GENERATING REPRODUCED ORDER- DEPENDENT REPRESENTATIONS OF A CHEMICAL COMPOUND

(71) Applicant: Collaborative Drug Discovery, Inc., Burlingame, CA (US)

(72) Inventors: Peter GEDECK, Arlington, VA (US); Barry A. BUNIN, Belmont, CA (US); Michael BOWLES, Redwood City, CA (US); Philip CHEUNG, San Diego, CA (US); Alex Michael CLARK, Montreal (CA)

(73) Assignee: Collaborative Drug Discovery, Inc., Burlingame, CA (US)

(21) Appl. No.: 17/709,614

(22) Filed: Mar. 31, 2022

Related U.S. Application Data
(60) Provisional application No. 63/172,303, filed on Apr. 8, 2021.

(51) Int. Cl. G16C 20/70; G16C 60/00; G16C 20/80

(52) U.S. Cl. CPC G16C 20/70; G16C 60/00; G16C 20/80

(57) ABSTRACT

A method includes generating a graph of a chemical compound based on at least one of an order-dependent representation of the chemical compound and a molecular graph representation of the chemical compound, encoding the graph based on an adjacency matrix of a graph convolutional neural network (GCN), an activation function of the GCN, and one or more weights of the GCN to generate a latent vector representation of the chemical compound, and decoding the latent vector representation based on a plurality of hidden states of a neural network (NN) to generate a reproduced order-dependent representation of the chemical compound.

### CROSS-REFERENCE TO RELATED APPLICATIONS

This application claims priority to and the benefit of U.S. Provisional Patent Application No. 63/172,303, filed on Apr. 8, 2021. The disclosure of the above application is incorporated herein by reference.

### GOVERNMENT LICENSE RIGHTS

This invention was made with government support under TR002527 awarded by the National Institutes of Health. The government has certain rights in the invention. 37 CFR 401.14(f)(4).

### FIELD

The present disclosure relates to systems and methods for generating reproduced order-dependent representations of chemical compounds.

### BACKGROUND

The statements in this section merely provide background information related to the present disclosure and may not constitute prior art.

Chemical compounds may be represented using various notations and nomenclatures, such as an order-dependent representation (e.g., a simplified molecular-input line-entry system (SMILES) string), an order-independent representation (e.g., a Morgan Fingerprint), or a molecular graph representation. In some forms, autoencoder/decoder networks may be implemented to encode/convert the order-dependent representations into a numerical representation (e.g., a latent vector) and subsequently decode the numerical representation back into the order-dependent representations. However, multiple latent vectors may be generated for a given order-dependent representation, thereby making it difficult to train a predictive model that utilizes latent vectors to predict one or more properties of a given chemical compound.

### SUMMARY

This section provides a general summary of the disclosure and is not a comprehensive disclosure of its full scope or all of its features.

The present disclosure provides a method that includes generating a graph of a chemical compound based on at least one of an order-dependent representation of the chemical compound and a molecular graph representation of the chemical compound, encoding the graph based on at least one of an adjacency matrix of a graph convolutional neural network (GCN), one or more characteristics of the graph, one or more activation functions of the GCN, and one or more weights of the GCN to generate a latent vector representation of the chemical compound, and decoding the latent vector representation based on a plurality of hidden states of a neural network (NN) to generate a reproduced order-dependent representation of the chemical compound.

In one form, the reproduced order-dependent representation is a simplified molecular-input line-entry system (SMILES) string associated with the chemical compound. In one form, the method includes identifying one or more fragments and one or more substructures of at least one of the order-dependent representation and the molecular graph representation, generating one or more nodes based on the one or more substructures, and generating one or more edges based on the one or more fragments, where the graph is further based on the one or more nodes and the one or more edges. In one form, the NN includes at least one of a gated recurrent unit, a long short-term memory (LSTM) unit, and an attention mechanism. In one form, the method includes training a machine learning model based on at least one of the order-dependent representation and the reproduced order-dependent representation, where the machine learning model includes the GCN and the NN. In one form, the method includes generating a molecular fingerprint of the chemical compound based on the latent vector representation and training the machine learning model based on at least one of the molecular fingerprint, the latent vector representation, and a loss function. In one form, the molecular fingerprint is a Morgan Fingerprint of the chemical compound. In one form, the method includes determining one or more statistical properties of the latent vector representation and training the machining learning model based on the one or more statistical properties.

The present disclosure provides a system for generating an input representing a chemical compound, where a machine learning model is configured to predict one or more properties of the chemical compound based on the input. The system includes one or more processors and one or more nontransitory computer-readable mediums storing instructions that are executable by the one or more processors. The instructions include generating a graph of a chemical compound based on at least one of an order-dependent representation of the chemical compound and a molecular graph representation of the chemical compound, encoding the graph based on an adjacency matrix of a graph convolutional neural network (GCN), an activation function of the GCN, and one or more weights of the GCN to generate a latent vector representation of the chemical compound, decoding the latent vector representation based on a plurality of hidden states of a recurrent neural network (RNN) to generate a reproduced order-dependent representation of the chemical compound, and training the machine learning model based on the order-independent representation, where the machine learning model includes the GCN and the RNN, and where the machine learning model is configured to predict one or more properties of the chemical compound based on the input. In one form, the instructions include encoding the graph based on one or more node aggregation functions of the GCN. In one form, the latent vector representation of the chemical compound is order independent.

The present disclosure provides a method including generating a latent vector based on a molecular graph representation of the chemical compound and decoding the latent vector representation based on a plurality of hidden states of a neural network to generate a token-based representation of the chemical compound. In one form, the token-based representation is a simplified molecular-input line-entry system (SMILES) string associated with the chemical compound. In one form, the method includes encoding the latent vector with latent vector conditioning based on an encoding routine and an embedding routine.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1A illustrates a functional block diagram of a chemical compound system in accordance with the teachings of the present disclosure;

FIG. 1B illustrates a functional block diagram of a trained chemical compound system in accordance with the teachings of the present disclosure;

FIG. 2 illustrates a molecular graph representation and an order-dependent representation of a chemical compound in accordance with the teachings of the present disclosure;

FIG. 3 illustrates a graph of a chemical compound in accordance with the teachings of the present disclosure;

FIG. 4 illustrates a graph convolutional neural network in accordance with the teachings of the present disclosure;

FIG. 5A illustrates an example neural network in accordance with the teachings of the present disclosure;

FIG. 5B illustrates another example neural network in accordance with the teachings of the present disclosure;

FIG. 5C illustrates an additional example neural network in accordance with the teachings of the present disclosure; and

FIG. 6 is a flowchart of an example control routine in accordance with the teachings of the present disclosure.

The drawings described herein are for illustration purposes only and are not intended to limit the scope of the present disclosure in any way.

### DESCRIPTION

The following description is merely exemplary in nature and is not intended to limit the present disclosure, application, or uses. It should be understood that throughout the drawings, corresponding reference numerals indicate like or corresponding parts and features.

The present disclosure provides systems and methods for generating a unique input representing a chemical compound and predicting, using a machine learning model, one or more properties of the chemical compound based on the input. To generate the unique input, the chemical compound system is trained to convert the input into a graph representing the chemical compound, encode the graph using a graph convolutional neural network to generate a latent vector representation of the chemical compound, and decode the latent vector representation based on a plurality of hidden states of a recurrent neural network to generate a reproduced order-dependent representation of the chemical compound.

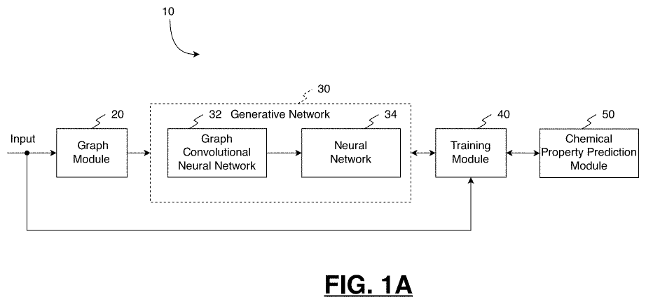

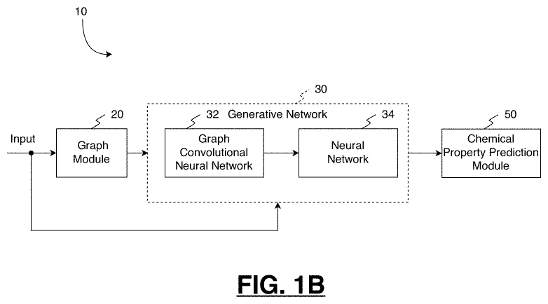

Referring to FIGS. 1A-1B, a functional block diagram of a chemical compound system 10 is shown and generally includes a graph module 20, a generative network 30, a training module 40, and a chemical property prediction module 50. While the components are illustrated as part of the chemical compound system 10, it should be understood that one or more components of the chemical compound system 10 may be positioned remotely from the chemical compound system 10. In one form, the components of the chemical compound system 10 are communicably coupled using a wired communication protocol and/or a wireless communication protocol (e.g., a Bluetooth®-type protocol, a cellular protocol, a wireless fidelity (Wi-Fi)-type protocol, a near-field communication (NFC) protocol, an ultra-wideband (UWB) protocol, among others).

Referring to FIG. 1A, a functional block diagram of the chemical compound system 10 is shown operating during a training mode (i.e., the chemical compound system 10 includes the training module 40). In FIG. 1B, a functional block diagram of the chemical compound system 10 is shown during the chemical property prediction mode (i.e., the chemical compound system 10 is sufficiently trained and, as such, the training module 40 is removed from chemical compound system 10).

In one form, the graph module 20 receives an input corresponding to at least one of an order-dependent representation of the chemical compound and a molecular graph representation of the chemical compound. As used herein, “order-dependent representation” refers to a nonunique text representation that defines the structure of the chemical compound. As an example, the order-dependent representation is a simplified molecular-input line-entry system (SMILES) string associated with the chemical compound, a DeepSMILES string, or a self-referencing embedded (SELFIE) string. As used herein, a “SMILES string” refers to a line notation that describes the corresponding structure using American Standard Code for Information Interchange (ASCII) strings. In one form, the SMILES string may be one of a canonical SMILES string (i.e., the elements are the string are ordered in accordance with one or more canonical rules) and/or an isomeric SMILES string (i.e., the string defines isotopes, chirality, double bonds, and/or other properties of the chemical compound). It should be understood that the graph module 20 may receive other text-based representations of the chemical compound (e.g., a systematic name, a synonym, a trade name, a registry number, and/or an international chemical identifier (InChI)), and subsequently converted to an order-dependent representation based on, for example, a table that maps one or more-order dependent representations and the text-based representations.

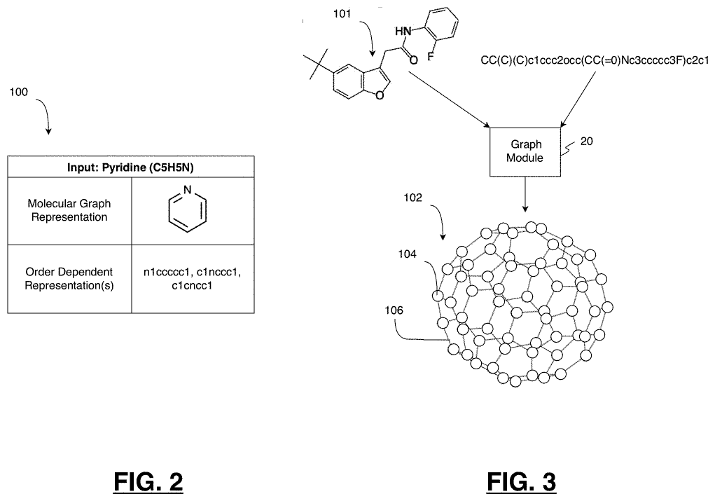

As used herein, the “molecular graph representation of the chemical compound” is a two-dimensional (20) molecular graph that represents three-dimensional (3D) information of the chemical compound, such as atomic coordinates, bond angles, and chirality. In one form, the 2D molecular graph is a tuple of a set of nodes and edges, where each edge connects pairs of nodes, and where each node is the set of all atoms of the chemical compound. As an example and as shown in FIG. 2, the graph module 20 receives and/or generates an input 100 that is one of a molecular graph and/or order-dependent representation of pyridine. To perform the functionality described herein, the graph module 20 may include one or more interface elements (e.g., audio input and natural language processing systems, graphical user interfaces, keyboards, among other input systems) operable by the user to generate an input representing a given chemical compound.

In one form and referring to FIGS. 1A-1B, the graph module 20 generates a graph of the chemical compound based on the input (i.e., at least one of the order-dependent representation and the molecular graph representation). As an example, the graph module 20 identifies one or more fragments and one or more substructures of the input. The one or more fragments of the input may include any fragment of the input, such as fragments connected to ring molecules of the input (e.g., monocycles or polycycles), fragments connected to amide bonds, fragments that identify a protein, fragments representing polymers or monomers, among others. The one or more substructures may include one or more combinations of fragments of the molecules, such as substituents and/or a moiety that collectively form a functional group.

Subsequently, the graph module 20 generates one or more nodes based on the substructures and one or more edges based on the one or more fragments, where the one or more nodes and one or more edges collectively form the graph. As a specific example and as shown in FIG. 3, the graph module 20 converts the SMILES string of 2-(5-tert-Butyl-1-benzofuran-3-yl)-N-(2-fluorophenyl)acetamide (e.g., CC(CXC)c1ccc2occ(CC(═O)Nc3ccccc3F)c2c1) or a corresponding molecular graph-based representation 101 to a graph 102 having a plurality of nodes 104 and edges 106. To perform the functionality described herein, the graph module 20 may perform known SMILES string to graph conversion routines that generate the graph 102 based on identified fragments and substructures of the SMILES string.

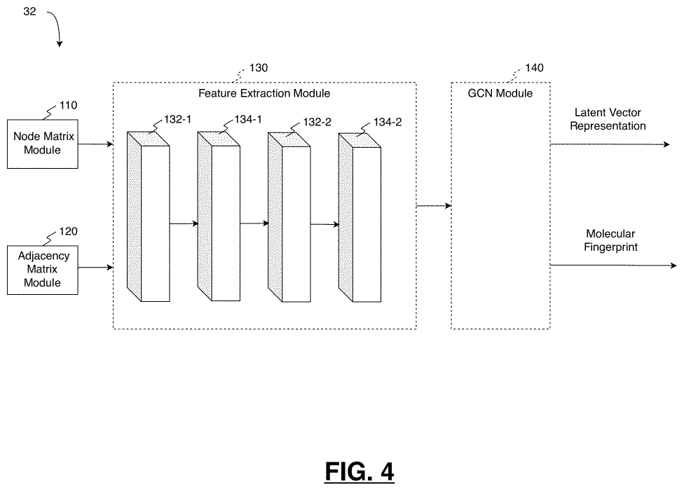

In one form and referring to FIGS. 1 and 4, the generative network 30 includes a graph convolutional neural network (GCN) 32 and a neural network 34. In one form, the GCN 32 includes a node matrix module 110, an adjacency matrix module 120, a feature extraction module 130, and a GCN module 140. In one form, the GCN 32 encodes the graph 102 based on at least one of a characteristic of the graph 102, an adjacency matrix defined by the node adjacency matrix module 120, one or more node aggregation functions and an activation function performed by the feature extraction module 130, and one or more weights of the feature extraction module 130 to generate a latent vector representation of the chemical compound.

In one form, the node matrix module 110 defines a node matrix based on the nodes 104 of the graph 102. As an example, the node matrix defines various atom features of the nodes 104, such as the atomic number, atom type, charge, chirality, ring features, hybridization, hydrogen bonding, aromaticity, among other atom features. To perform the functionality described herein, the node matrix module 110 may perform known input featurization routines to encode the atom features of the nodes 104 into the node matrix. In one form, the adjacency matrix module 120 defines an adjacency matrix based on the edges 106 of the graph 102. In one form, the adjacency matrix is an k×k matrix, where k is equal to the number of nodes 104, and where each element of the adjacency matrix indicates whether one of the edges 106 connects a given pair of nodes 104 of the graph 102.

In one form, the feature extraction module 130 includes convolutional layers 132-1, 132-2 (collectively referred to hereinafter as “convolutional layers 132”) and activation layers 134-1, 134-2 (collectively referred to hereinafter as “activation layers 134”). While two convolutional layers 132 and two activation layers 134 are shown, it should be understood that the feature extraction module 130 may include any number of convolutional layers 132 and activation layers 134 in other forms and is not limited to the example described herein. It should also be understood that the feature extraction module 130 may also include other layers that are not shown, such as one or more pooling layers.

In one form, the convolutional layers 132 are configured to perform a graph convolutional operation based on the node matrix and the adjacency matrix. As an example, at least one of the convolutional layers 132 performs one or more node aggregation functions, which comprise selecting an element from the node matrix corresponding to one of the nodes 104 and determining the atom features associated with the given node 104 and connected nodes (as defined by the adjacency matrix). The node aggregation function may also include performing a convolutional operation on the atom features associated with the given node 104 and the connected nodes to form a linear relationship between the given node 104 and the connected nodes and performing a pooling operation (e.g., a downsampling operation) to adjust the resolution of the linear relationship and generate one or more atom feature outputs. It should be understood that the node aggregation function may be performed for any number of elements of the node matrix (e.g., each element of the node matrix). As another example, at least one of the convolutional layers 132 performs an edge weight filtering routine that includes applying an edge feature matrix to at least one of the node matrix and the adjacency matrix, where the edge feature matrix defines one or more weights that selectively filter/adjust the atom feature values of the node matrix and/or adjacency matrix.

In one form, the activation layers 134 are configured to perform an activation function on the one or more atom feature outputs of the convolutional layers 132 to learn one or more features of the nodes 104. Example activation functions include, but are not limited to, a sigmoid activation function, a tan h activation function, a rectified linear unit function, among others.

In one form, the GCN module 140 encodes the graph 102 into a latent vector representation by combining the one or more learned features associated with each of the nodes 104. As an example, the GCN module 140 performs known transformation operations to sum the one or more learned features associated with each of the nodes 104 and generate a fixed-size descriptor vector or a scale-invariant feature (SIFT) vector (as the latent vector representation). In one form, the latent vector representation is an order-independent representation of the chemical compound. As used herein, “order-independent representation” refers to a uniquely defined textual or numerical representation of the structure of the chemical compound that is independent of any arbitrary ordering of the atoms. In one form, the latent vector representation may also correspond to a given set of chemical and/or biological properties.

In one form, the GCN module 140 generates a molecular fingerprint of the chemical compound based on the latent vector representation of the chemical compound and known latent vector to molecular fingerprint conversion routines. Example molecular fingerprints include, but are not limited to: a Morgan fingerprint, a hashed-based fingerprint, an atom-pair fingerprint, among other known molecular fingerprints. As described below in further detail, the training module 40 is configured to train the GCN 32 and/or the neural network 34 based on the molecular fingerprint and/or the latent vector representation.

In one form, the neural network 34 is a recurrent neural network, but it should be understood that the neural network 34 may employ a convolutional neural network in other forms. The neural network 34 decodes the latent vector representation generated by the GCN 32 based on a plurality of hidden states of the recurrent neural network to generate a reproduced order-dependent representation of the chemical compound.

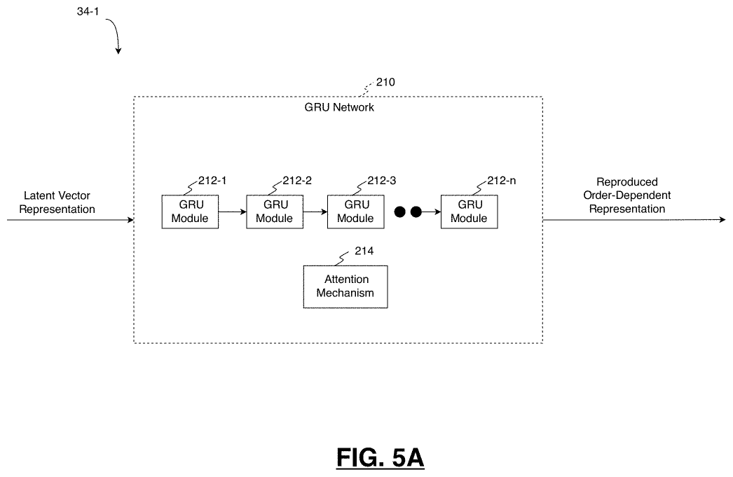

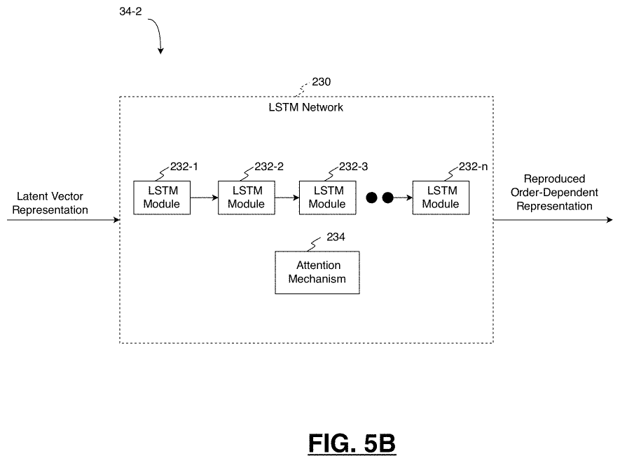

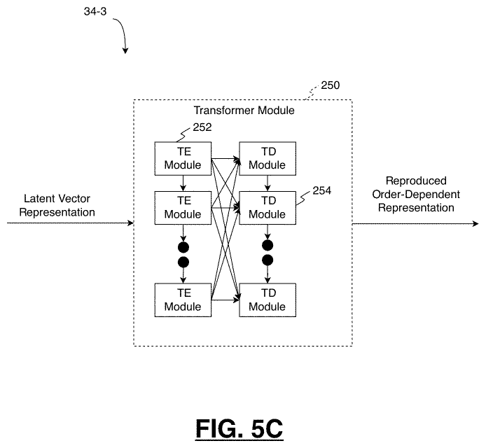

As an example and as shown in FIG. 5A, neural network 34-1 (as the neural network 34) is a gated recurrent unit (GRU) network 210 and includes gated recurrent unit modules 212-1, 212-2, 212-3, . . . 212-n (collectively referred to hereinafter as “GRU modules 212”) and an attention mechanism 214. It should be understood that the GRU network 210 may include any number of GRU modules 212 in other forms and is not limited to the example described herein. It should also be understood that the attention mechanism 214 may be removed from the GRU network 210. Furthermore, it should be understood that the GRU modules 212 may be replaced with a plurality of ungated recurrent units (not shown) in other forms.

In one form, each of the GRU modules 212 generates an output vector (hᵥ₊₁) based on an update gate vector (zᵥ), a reset gate vector (rᵥ), a hidden state vector (h′ᵥ), and the following relations:

zᵥ=σ(W_{z}xᵥ+U_{z}aᵥ+V_{z}cᵥ+b_{z}) (1)

rᵥ=σ(Wᵣxᵥ+Uᵣaᵥ+Vᵣcᵥ+bᵣ) (2)

h′ᵥ=tan h(W(rᵥ⊙hᵥ)+Uaᵥ+Vcᵥ+bₕ) (3)

hᵥ₊₁=(1−zᵥ)⊙hᵥ+zᵥ⊙h′ᵥ (4)

In relations (1)-(4), W_{z}, Wᵣ, U_{z}, and Uᵣ are input weights of the update gate vector and reset gate vectors, W is a weight of the GRU module 212, xᵥ is an input representing one or more elements of the latent vector, aᵥ is a hidden state value (i.e., the reset gate vector depends on the hidden state of the preceding GRU module 212), cᵥ is a conditioning value, b_{z}, bᵣ, bₕ are bias values, V are matrices that are based on a predefined hidden dimension and the latent vector representation, and a is a sigmoid function. In one form, the update gate vector indicates whether the GRU module 212 updates and/or preserves the hidden state value, and the reset gate vector indicates whether the GRU module 212 utilizes the previous hidden state value to calculate the hidden state vector and the output vector.

Specifically, the GRU modules 212 decode the latent vector representation based on the hidden states of the GRU modules 212 to generate a token-based representation of the chemical compound having one or more tokens. As used herein, “tokens” refer to one or more characters of the order-dependent representation, such as one or more characters of the SMILES string. In one form, the GRU modules 212 decode the latent vector representation and generate the token-based representation of the chemical compound one token at a time.

As an example, the first GRU module 212-1 generates the first token based on the latent vector representation and a trainable starting state, and the first token may be a beginning-of-sequence (BOS) token that initiates the GRU modules 212. In some forms, the first GRU module 212-1 is further configured to encode the latent vector representation with latent vector conditioning routine based on an encoding routine (e.g., one-hot encoding routine) and an embedding routine, thereby enabling the first GRU module 212-1 to initialize the hidden state of the GRU modules 212. After producing the first token, the second GRU module 212-2 generates a second token based on the hidden state of the first GRU module 212-1 and the latent vector representation. After producing the second token, the third GRU module 212-3 generates a third token based on the hidden state of the second GRU module 212-2 and the latent vector representation. The GRU modules 212 collectively and recursively generate tokens until the last GRU module 212-n produces an end-of-sequence (EOS) token. In one form, the GRU module 212-n aggregates each of the generated tokens to generate the reproduced order-dependent representation of the chemical compound.

In one form, the attention mechanism 214 instructs each of the GRU modules 212 to generate the respective token based on each previous hidden states. As an example and after producing the second token, the third GRU module 212-3 generates a third token based on the hidden state of the first and second GRU modules 212-1, 212-2 and the latent vector representation. As another example, the nth GRU module 212-n generates the EOS token based on the hidden state of each of the preceding GRU modules 212 and the latent vector representation.

As another example and as shown in FIG. 5B, neural network 34-2 (as the neural network 34) is a long short-term memory (LSTM) network 230 and includes LSTM modules 232-1, 232-2, 232-3 . . . 232-n (collectively referred to hereinafter as “LSTM modules 232”) and an attention mechanism 234. It should be understood that the LSTM network 230 may include any number of LSTM modules 232 in other forms and is not limited to the example described herein. In one form, the LSTM modules 232 are configured to perform similar functions as the GRU modules 212, but in this form, LSTM modules 232 are configured to calculate input vectors, output vectors, and forget vectors based on the hidden states of the LSTMs and the latent vector representation to generate the reproduced order-dependent representation of the chemical compound. In one form, the attention mechanism 234 is configured to perform similar operations as the attention mechanism 214 described above.

As an additional example and as shown in FIG. 5C, neural network 34-3 (as the neural network 34) is a transformer 250 and includes transformer encoder modules 252-1, 252-2, . . . 252-n (collectively referred to hereinafter as “TE modules 252”) and transformer decoder modules 254-1, 254-2, . . . 254-n (collectively referred to hereinafter as “TD modules 254”). In one form, the TE modules 252 each include feed-forward and self-attention layers that are collectively configured to encode a portion of the latent vector representation. The TD modules 254 each include feed-forward, self-attention, and encoder-decoder attentional layers that collectively decode each of the encoded latent vector representation portions generated by the TE modules 252 to generate the reproduced order-dependent representation of the chemical compound.

In one form, the training module 40 is configured to train a machine learning model (e.g., the generative network 30 and/or the chemical property prediction module 50) based on at least one of the input, the reproduced order-dependent representation, the latent vector representation, and the molecular fingerprint. As an example, the training module 40 is configured to determine an aggregate loss value based on a loss function that derives the difference between, for example, the input and the reproduced order-dependent representation and/or the input and the molecular fingerprint. In some forms, the loss function includes a regularization variable that prevents memorization and overfitting problems associated with larger weights of the GCN 32 and/or the neural network 34. Accordingly, the training module 40 may iteratively adjust one or more weights of the feature extraction module 130 of the GCN 32 and/or one or more weights of the neural network 34 (e.g., the weights of the GRU modules 212) until the aggregate loss value associated is less than a threshold value.

As another example, the training module 40 instructs the chemical property prediction module 50 to determine one or more statistical properties of the latent vector representation, such as a water-octanal partition coefficient (log P), a synthetic accessibility score (SAS), a qualitative estimate of drug-likeness (QED), a natural-product (NP) score, among other statistical properties of the latent vector representation. The training module 40 may determine an aggregate loss value based on a loss function that quantifies the difference between the determined statistical properties and known statistical properties associated with the input. Accordingly, the training module 40 may iteratively adjust one or more weights of the feature extraction module 130 of the GCN 32 and/or one or more weights of the neural network 34 (e.g., the weights of the GRU modules 212) until the aggregate loss value associated with the statistical properties is less than a threshold value.

In one form, the chemical property prediction module 50 predicts a chemical property of the chemical compound based on the reproduced order-dependent representation and/or the latent vector representation. In one form, the chemical property prediction module 50 employs known multilayer perceptron networks and/or a regression model that predict the chemical properties of the chemical compound based on the reproduced order-dependent representation and/or the latent vector representation.

As an example, the chemical property prediction module 50 predicts one or more statistical properties of the latent vector representation (as the chemical property) while training the GCN 32 and/or the neural network 34. As another example, when the GCN 32 and the neural network 34 are trained (i.e., the input corresponds to the reproduced order-dependent representation of the input generated by the generative network 30), the chemical property prediction module 50 may predict various chemical properties of the input, generate/identify new chemical compounds that are related to the input, and/or filter chemical compounds that are unrelated to the input and/or have a statistical property that deviates from the input beyond a threshold amount.

Accordingly, when the chemical property prediction module 50 and the generative network 30 are trained, the amount of time needed for a medicinal chemist to modify a chemical compound and generate a lead compound to achieve a desired level of potency and other chemical/pharmacological properties (e.g., absorption, distribution, metabolism, excretion, toxicity, among others) during drug discovery is substantially reduced. As such, the trained chemical property prediction module 50 and the generative network 30 enables medicinal chemists can select lead candidate series explore chemical space similar to the chemical compound more effectively, reduces failure rates for chemical compounds that advance through the drug discovery process, and accelerate the drug discovery process.

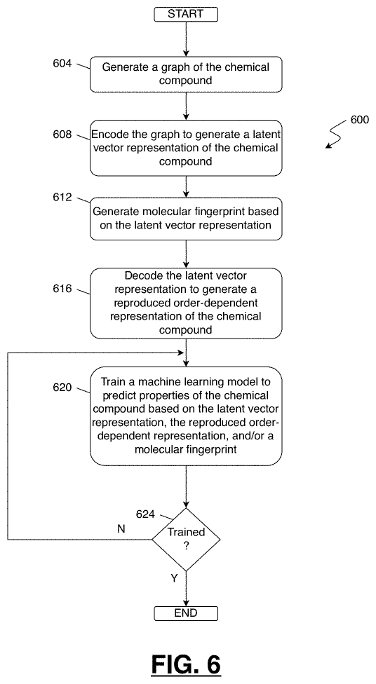

Referring to FIG. 6, a routine 600 for defining a machine learning model configured to predict one or more properties associated with a chemical compound is shown. At 604, the graph module 20 generates a graph of the chemical compound. At 608, the generative network 30 encodes the graph to generate a latent vector representation of the chemical compound. At 612, the generative network 30 generates a molecular fingerprint based on the latent vector representation. At 616, the generative network 30 decodes the latent vector representation to generate a reproduced order-dependent representation of the chemical compound. At 620, the training module 40 trains a machine learning model (i.e., the chemical property prediction module 50 and/or the generative network 30) to predict properties of the chemical compound based on the latent vector representation, the reproduced order-dependent representation, and/or the molecular fingerprint. At 624, the training module 40 determines whether the machine learning model is trained based on the loss function. If the machine learning model is trained, the routine ends. Otherwise, the routine 600 proceeds to 620.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. This modification is desired for various reasons including industrial practice; material, manufacturing, and assembly tolerances; and testing capability.

As used herein, the phrase at least one of A, B, and C should be construed to mean a logical (A OR B OR C), using a non-exclusive logical OR, and should not be construed to mean “at least one of A, at least one of B, and at least one of C.”

The description of the disclosure is merely exemplary in nature and, thus, variations that do not depart from the substance of the disclosure are intended to be within the scope of the disclosure. Such variations are not to be regarded as a departure from the spirit and scope of the disclosure.

In the figures, the direction of an arrow, as indicated by the arrowhead, generally demonstrates the flow of information (such as data or instructions) that is of interest to the illustration. For example, when element A and element B exchange a variety of information, but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, the term module may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality, such as, but not limited to, transceivers, routers, input/output interface hardware, among others; or a combination of some or all of the above, such as in a system-on-chip.

The term memory is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The term code, as used below, may include software, firmware, and/or microcode, and may refer to computer programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As an example, source code may be written using syntax from languages including C, C++, C#, Objective-C, Swift, Haskell, Go, SQL, R, Lisp, Java®, Fortran, Perl, Pascal, Curd, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

1. A method comprising:
generating a graph of a chemical compound based on at least one of an order-dependent representation of the chemical compound and a molecular graph representation of the chemical compound;
encoding the graph based on at least one of an adjacency matrix of a graph convolutional neural network (GCN), one or more characteristics of the graph, one or more activation functions of the GCN, and one or more weights of the GCN to generate a latent vector representation of the chemical compound; and
decoding the latent vector representation based on a plurality of hidden states of a neural network (NN) to generate a reproduced order-dependent representation of the chemical compound.

2. The method of claim 1, wherein the reproduced order-dependent representation is a simplified molecular-input line-entry system (SMILES) string associated with the chemical compound.

3. The method of claim 1 further comprising:
identifying one or more fragments and one or more substructures of at least one of the order-dependent representation and the molecular graph representation;
generating one or more nodes based on the one or more substructures; and
generating one or more edges based on the one or more fragments, wherein the graph is further based on the one or more nodes and the one or more edges.

4. The method of claim 1, wherein the NN includes at least one of a gated recurrent unit, a long short-term memory (LSTM) unit, and an attention mechanism.

5. The method of claim 1 further comprising training a machine learning model based on at least one of the order-dependent representation and the reproduced order-dependent representation, wherein the machine learning model includes the GCN and the NN.

6. The method of claim 5 further comprising:
generating a molecular fingerprint of the chemical compound based on the latent vector representation; and
training the machine learning model based on at least one of the molecular fingerprint, the latent vector representation, and a loss function.

7. The method of claim 6, wherein the molecular fingerprint is a Morgan Fingerprint of the chemical compound.

8. The method of claim 6 further comprising:
determining one or more statistical properties of the latent vector representation; and
training the machining learning model based on the one or more statistical properties.

9. The method of claim 1 further comprising encoding the graph based on one or more node aggregation functions of the GCN.

10. The method of claim 1 further comprising, wherein the latent vector representation of the chemical compound is an order independent representation.

11. A system for defining a machine learning model configured to predict one or more properties associated with a chemical compound, the system comprising:
one or more processors and one or more nontransitory computer-readable mediums storing instructions that are executable by the one or more processors, wherein the instructions comprise:
generating a graph of the chemical compound based on at least one of an order-dependent representation of the chemical compound and a molecular graph representation of the chemical compound;
encoding the graph based on an adjacency matrix of a graph convolutional neural network (GCN), one or more characteristics of the graph, one or more activation functions of the GCN, and one or more weights of the GCN to generate a latent vector representation of the chemical compound;
decoding the latent vector representation based on a plurality of hidden states of a recurrent neural network (RNN) to generate a reproduced order-dependent representation of the chemical compound; and
training the machine learning model based on the reproduced order-dependent representation, wherein the machine learning model includes the GCN and the RNN, and wherein the machine learning model is configured to predict one or more properties of the chemical compound.

12. The system of claim 11, wherein the instructions further comprise encoding the graph based on one or more node aggregation functions of the GCN.

13. The system of claim 11, wherein the latent vector representation of the chemical compound is an order independent representation.

14. The system of claim 11, wherein the reproduced order-dependent representation is a simplified molecular-input line-entry system (SMILES) string associated with the chemical compound.

15. The system of claim 11, wherein the instructions further comprise:
identifying one or more fragments and one or more substructures of at least one of the order-dependent representation and the molecular graph representation;
generating one or more nodes based on the one or more substructures; and
generating one or more edges based on the one or more fragments, wherein the graph is further based on the one or more nodes and the one or more edges.

16. The system of claim 11, wherein the RNN includes at least one of a gated recurrent unit, a long short-term memory (LSTM) unit, an ungated recurrent unit, and an attention mechanism.

17. The system of claim 11, wherein the instructions further comprise:
generating a molecular fingerprint of the chemical compound based on the latent vector representation; and
training the machine learning model based on at least one of the molecular fingerprint, the latent vector representation, the reproduced order-dependent representation, and a loss function.

18. A method comprising:
generating a latent vector based on a molecular graph representation of a chemical compound; and
decoding the latent vector representation based on a plurality of hidden states of a neural network to generate a token-based representation of the chemical compound.

19. The method of claim 18, wherein the token-based representation is a simplified molecular-input line-entry system (SMILES) string associated with the chemical compound.

20. The method of claim 18 further comprising encoding the latent vector with latent vector conditioning based on an encoding routine and an embedding routine.
